(19) United States
(12) Patent Application Publication
(10) Pub. No.: US 2010/0318019 A1
(43) Pub. Date: Dec. 16, 2010

(54) ELECTROPHYSIOLOGY DEVICES EMPLOYING ELECTRICALLY CONDUCTIVE POLYMER CONDUCTORS AND METHODS OF MANUFACTURING SUCH DEVICES

(75) Inventors: Elizabeth Nee, Chicago, IL (US); Scott Salys, Plano, TX (US); Greg Kampa, Castaic, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/484,539

(22) Filed: Jun. 15, 2009

(51) Int. Cl. A61N 1/05; H01B 3/44; A61N 1/30; A61M 25/00

(52) U.S. Cl. 604/21; 174/110 R; 607/116; 29/825

(57) ABSTRACT

A medical tubular body that may be used in an implantable medical lead, a catheter, a sheath and introducer is disclosed herein. The medical tubular body may include a tubular layer formed of an electrically insulating polymer and an electrically conductive polymer strip imbedded in and longitudinally extending through the insulating polymer.

### FIELD OF THE INVENTION

The present invention relates to medical apparatus and methods. More specifically, the present invention relates to electrophysiology devices, such as, for example, catheters, leads and delivery tools, and methods of using and manufacturing such devices.

### BACKGROUND OF THE INVENTION

Currently, when an electrophysiology device (e.g., a lead or treatment, diagnosis or delivery tool (e.g., a catheter, sheath or introducer)) is manufactured, wires are run through the length of the device to connect the electrodes on a distal end of the device to a connector on a proximal end of the device. Using wires creates some difficulties in assembly of a device, as the electrode wires, which are rather delicate, are threaded through the length of the device, which can be up to four feet.

Handling and assembly can damage the insulation on the wires. This insulation damage can lead to electrical opens or shorts. Depending on the construction of the device, there is also a chance the wires may rub against internal components. This rubbing can also cause electrical opens or shorts when trying to administer a treatment (e.g., electrotherapy) or to take measurements (e.g., for an electrogram).

New device designs are incorporating a greater number of electrodes. A greater number of electrodes results in a greater number of electrical wires extending through the device. To facilitate the device being able to accommodate the greater number of electrical wires, the electrical wires used for the device end up being smaller. As the electrical wires get smaller, it becomes increasingly difficult to attach them to the electrodes and the connector. Also, as the electrical wires get smaller, they also get more fragile, which results in assembly difficulties. An additional concern is that for some devices, such as, for example, sheaths and introducers, the device walls are so thin that it is difficult to create a lumen through which the electrode wires may be routed.

There is a need in the art for electrophysiology devices having an electrical conductor configuration that addresses the above-mentioned issues.

There is also a need in the art for a method of manufacturing such electrophysiology devices.

### BRIEF SUMMARY OF THE INVENTION

A medical tubular body that may be used in an implantable medical lead, a catheter, a sheath and introducer is disclosed herein. In one embodiment, the medical tubular body includes a tubular layer formed of an electrically insulating polymer and an electrically conductive polymer strip imbedded in and longitudinally extending through the insulating polymer.

A medical longitudinally extending body that may be used in an implantable medical lead, a catheter, a sheath and introducer is also disclosed herein. In one embodiment, the body includes a longitudinally extending portion of the body, the longitudinally extending portion formed of an electrically insulating polymer and an electrically conductive polymer strip imbedded in and longitudinally extending through the insulating polymer, the insulating polymer forming a majority of the longitudinally extending portion.

A medical longitudinally extending body that may be used in an implantable medical lead, a catheter, a sheath and introducer is also disclosed herein. In one embodiment, the body includes a longitudinally extending portion of the body and an electrically conductive strip. The longitudinally extending portion is formed of an electrically insulating polymer. The electrically conductive strip extends along an outer circumferential surface of the longitudinally extending portion of the body. The strip is deposited via at least one of vapor deposition, printing, and painting.

A method of manufacturing a medical longitudinally extending body is disclosed herein. In one embodiment, the method includes: providing an electrically insulating polymer; providing an electrically conductive polymer; and co-extruding the electrically insulating polymer and the electrically conductive polymer into a longitudinally extending portion of the medical longitudinally extending body, wherein the insulating polymer forms a majority of the longitudinally extending portion and the electrically conductive polymer forms a strip imbedded in and longitudinally extending through the insulating polymer.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a side view of an electrophysiology device and, more specifically, a passive-fixation bipolar endocardial body implantable lead.

FIG. 2 is a cross section of the tubular body as taken along section line A-A in FIG. 1.

FIG. 3 is an isometric view of the cross section of FIG. 2.

FIG. 4 is an isometric view of the wall structure similar to that of FIG. 2.

FIG. 5 is a cross section of an intermediate layer and an inner layer of the tubular body as taken along section line A-A in FIG. 1.

FIG. 6 is an isometric view of the intermediate layer of the tubular body in the same view as FIG. 5.

FIG. 7 is an isometric view of the wall structure of the tubular body in the same view as FIG. 7.

FIG. 8 is the same view as FIG. 7, except with an electrode in electrical communication with an electrically conductive strip.

FIG. 9 is generally the same view as depicted in FIG. 2, except of the entire wall structure of the tubular body as described with respect to FIG. 2-4.

FIGS. 10-13 are cross sections of a body similar to FIG. 2, except the body being formed with a core.

FIG. 14 is a longitudinal cross section of the tubular body.

FIG. 15 is isometric views of the body proximal end and a connector end.

FIGS. 16-18 are isometric views of the tubular body with strips formed of conductive deposition, the strips having a pattern.

### DETAILED DESCRIPTION

An electrophysiology device 10 is disclosed herein. Depending on the embodiment, the electrophysiology device 10 may be any type of tubular electrophysiology device 10 having a tubular body 12, including, for example and without limitation, leads, catheters, sheaths, introducers, etc. The device 10 may be configured to generally eliminate the use of wires in the tubular body 12 of the device 10. For example, in one embodiment, the device 10 may include a tubular body 12 having a tubular layer 31formed of an electrically insulating polymer and electrically conductive polymer strips 44 imbedded in and longitudinally extending through the insulating polymer. The electrically insulating polymer may insulate the strips 44 where the strips 44 are completely imbedded in the electrically insulating polymer. Alternatively, additional insulating materials may be applied about the tubular layer where the strips 44 are not completely imbedded in the insulating polymer.

In another embodiment, conductive depositions may be used to form the strips 44 on the outer circumferential surface of the tubular layer. Insulating materials may be applied about the tubular layer.

The following description presents preferred embodiments of the electrophysiology device representing the best mode contemplated for practicing the electrophysiology device. This description is not to be taken in a limiting sense but is made merely for the purpose of describing the general principles of the electrophysiology device, the scope of which is defined by the appended claims.

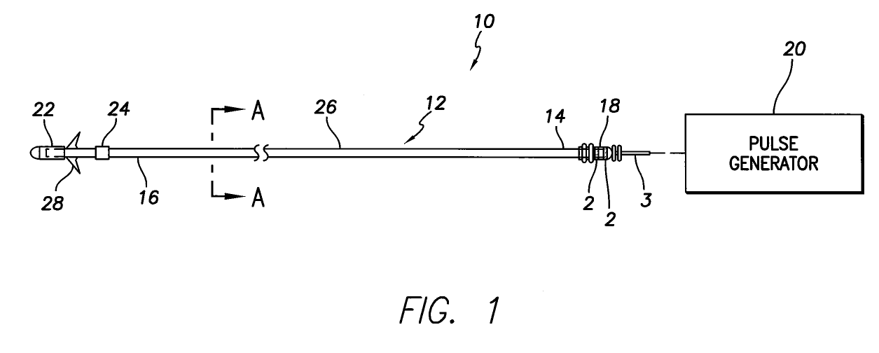

FIG. 1 is a side view of an electrophysiology device 10 and, more specifically, a passive-fixation bipolar endocardial body implantable lead 10. While the following discussion of FIG. 1 is given in the context of the electrophysiology device 10 being a lead 10, it should be understood that the inventive concepts described in this Detailed Description and recited in the appended claims are readily applicable to most, if not all, tubular body type electrophysiology devices, including, without limitation, those types of devices having a tubular body 12 such as, for example, leads, catheters, sheaths, introducers, etc.

As shown in FIG. 1, the lead 10 includes a tubular body 12 having a proximal end portion 14 and a distal end portion 16. The proximal end portion 14 of the tubular body 12 carries a connector assembly 18, conforming in this example to the IS-1 standard, for coupling the tubular body 12 to a receptacle on a pulse generator 20 such as, for example, a pacemaker or an implantable cardioverter/defibrillator (“ICD”). The distal end portion 16 of the tubular body 12 carries a tip electrode 22 and a ring electrode 24 proximal of the tip electrode and spaced apart therefrom. The ring electrode 24 may serve as a pacing/sensing electrode, although it will be evident that it may instead function as a cardioverting and/or defibrillating electrode. While the lead 10 depicted in FIG. 1 is depicted as a passive fixation lead, in other embodiments, the lead 10 may be configured for active fixation, even being equipped at the distal end with a helix anchor or other type of active fixation feature.

The lead connector end 18 may include one or more ring contacts 2 and a pin contact 3, the contacts 2, 3 contacting complementary contacts in the pulse generator 20 when the lead connector end 18 is received in the pulse generator 20. The tubular body 12 may be adapted to transmit stimulating and/or sensed electrical signals between the connector assembly 18, on the one hand, and the tip and the ring electrodes 22 and 24, on the other.

By way of example and not limitation, the distal end portion 16 of the tubular body 12 of the lead 10 may have a diameter of about 0.026 inch (2F) to about 0.131 inch (10F), with a diameter of about 0.079 (6F) being preferred, and the ring electrode 24, where it serves a sensing function, may have a diameter of about 0.079 inch (6F) and a length of about 0.100 inch. The tubular body 12 may include a tubular insulating sheath or housing 26 of a suitable insulative biocompatible biostable material such as, for example, silicone rubber, polyurethane or other suitable elastomer, extending the entire length of the tubular body 12. The housing 26 may include along the distal end portion of the lead a plurality of rearwardly projecting tines 28 functioning, as is well know in the art, to interlock in the trabeculae within the heart and thereby prevent displacement of the distal end portion 16 once the lead 10 is implanted. Although tines are the preferred anchoring features for purposes of the present lead 10, it will be understood by those skilled in the art that fins, a screw-in helix, or some other suitable active fixation anchoring features may be used instead. Also, the lead may be configured for passive fixation via, for example, one or more S-shaped bends in the tubular body 12 along the distal end portion, and may be without tines or active fixation features. The S-shaped bends may bias against the walls of the coronary sinus region to maintain the lead 10 in position.

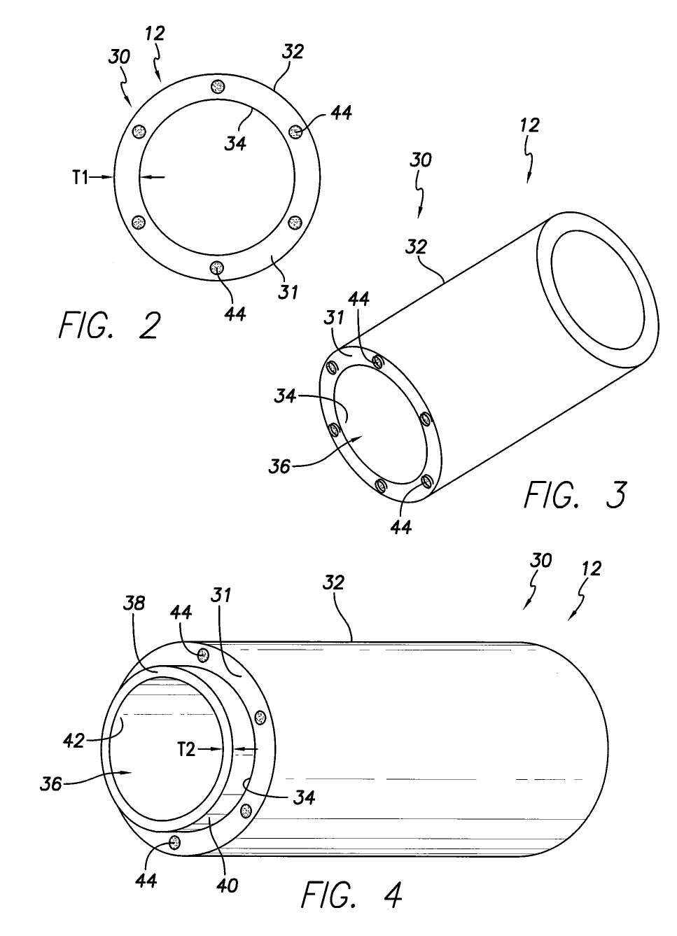

For a detailed discussion regarding a first configuration of the wall structure 30 of the tubular body 12, reference is made to FIG. 2, which is a cross section of the tubular body 12 as taken along section line A-A in FIG. 1. As shown in FIG. 2, in some embodiments, the tubular body 12 includes a wall structure 30 including a first layer 31 having an outer circumferential surface 32, an inner circumferential surface 34 and a thickness T1. In some embodiments, as shown in FIG. 3, which is an isometric view of the cross section of FIG. 2, the wall structure 30 of the tubular body 12 may be limited to the first layer 31depicted in FIG. 2 and 3. In such an embodiment, the inner circumferential surface may 34 may define a central lumen 36.

In other embodiments, as depicted in FIG. 4, which is a view similar to FIG. 3, the wall structure 30 may include other layers in addition to the first layer 31, wherein additional layers of the wall structure 30 extend over and/or under the first layer 31. For example, as illustrated in FIG. 4, the first layer 31 may extend over an additional layer 38. Such an additional layer 38 of the wall structure 30 may be a helically wound coil, a braid layer, or a polymer layer formed of a polymer material different from the polymer material forming the first layer 31. The additional or second layer 38 may include an outer circumferential surface 40, an inner circumferential surface 42, and a thickness T2. The inner circumferential surface 42 of the second layer 38 may define the central lumen 36.

As can be understood from FIGS. 2-4, in one embodiment, the first layer 31 includes electrical conductors 44 longitudinally extending through the thickness T1 of the first layer 31. In one embodiment, the electrical conductors 44 are in the form of strips 44 of electrically conductive polymer material generally completely imbedded in the material forming the bulk of the first layer 31. For example, the first layer 31 may be formed of polyether block amide (“PEBAX”) with the electrically conductive polymer strips 44 being coextruded along with the PEBAX forming the bulk of the first layer 31. Thus, the PEBAX material forming the bulk material of the first layer surrounding the electrically conductive polymer strips 44 may electrically isolate the strips 44 from each other and external structures or conditions that may cause a strip 44 to electrically short. As described below, additional layers extending below and/above the first layer 31 may provide additional electrical insulation for the strips 44.

The electrically conductive polymer strips 44 may be formed of electrically conductive silicone rubber, epoxy, adhesive, etc. As discussed in greater detail below, to access the conductive strips 44, for example, to allow for an electrical connection between the strips 44 and an electrode 24 or contact ring of a connector end 18, the PEBAX of the first layer 31 may be cut away (e.g., via mechanical, laser, chemical or other cutting processes) or otherwise removed over the strips 44 in those areas needed to allow for the electrical connection.

In one embodiment, as indicated in FIG. 4, the first layer 31 with its integral co-extruded electrically conductive polymer strips 44 may be extruded over an inner layer 38 or co-extruded with an inner layer 38, wherein the inner layer 38 may be, for example, a PEBAX layer, a polytetrafluoroethylene (“PTFE”) inner tube, a braided layer, or etc.

In one embodiment, the first layer 31 with its integral coextruded electrically conductive polymer strips 44 may be pulled over an inner layer 38, which may be a PTFE inner tube, a braided layer, or etc. A fluorinated ethylene propylene (“FEP”) heat shrink tube may be pulled over the outer circumferential surface 32 of the first layer 31, and the entire assembly may be subjected to a heat shrink process, wherein the PEBAX forming the first layer 31 is caused to reflow to adhere to the inner layer 38, in the case of a PTFE inner layer 38, or impregnate the inner layer 38, in the case of a braided layer 38.

While PEBAX may be used for the first layer 31, in other embodiments, the first layer 31 may be other polymer layers such as, for example, polyurethane, silicone rubber-polyurethane-copolymer (“SPC”), nylon, etc. Also, in some embodiments, the inner layer 38 may be formed of multiple layers itself. For example the inner layer 38 may be formed of an inner most layer formed of a PTFE tube surrounded by an outer braid layer, and this composite inner layer 38 may then be surrounded by the PEBAX outer layer 31, which may be reflowed about the composite inner layer 38.

In one embodiment, as depicted in FIG. 9, which is a cross section similar to FIG. 2, once the wall structure 30 of the tubular body 12 is assembled as discussed with respect to FIGS. 2-4, a portion of the thickness T1 is removed from the first layer 31 to accommodate the placement of an electrode 24 or contact ring of a connector end 18 in a configuration where the tubular body 12 is generally isodiametric, the outer circumferential surface 60 of the lead body 12 being generally continuous and consistent with respect to diameter. In the vicinity of one of the electrically conductive strips 44, the first layer 31 is removed in its entirety, as indicated by arrow B. In such an embodiment, the outer circumferential surface 32 (see FIG. 4) of the first layer 31 in combination with the outer circumferential surface 62 (see FIG. 9) of the electrode 24 may form the outer circumferential surface 60 (see FIG. 9) of the tubular body 12. Also, the remaining portion of the first layer 31 may serve to electrically isolate the electrode 24 from all of the strips 44, except the strip 44 exposed at arrow B, which may be in electrical contact with the strip 44 via an extension 64 of the electrode 24 that extends through the first layer 31 to contact the strip 44.

In another embodiment, as can be understood from FIG. 9, the wall structure 30 of the tubular body 12 is assembled as described above with respect to FIG. 4. The first layer 31 remains in its entirety, except in a location over one of the strips 44, as indicated by arrow B. An electrode 24 is mounted over the outer circumferential surface of the first layer 31 such that the first layer 31 electrically isolates the electrode 24 from all of the strips 44, except the exposed strip 44 at arrow B, which is in electrical contact with the extension 64 of the electrode 24 that extends through the opening made in the first layer 31. To make the tubular body 12 generally isodiametric, another layer may be extended about the first layer 50 generally everywhere not occupied by the electrode 24, the outer circumferential surfaces of the electrode 24 and the another layer forming the outer circumferential surface 60 of the tubular body 12 and acting as a jacket. In such an embodiment, the jacket layer may be formed of silicone rubber, SPC, polyurethane, etc.

In one embodiment, the electrode 24 or contact of the connector end 18 may be in the form of a ring, partial ring, button or other configuration. The electrode 24 or contact of the connector end 18 may be formed of an electrically conductive metal (e.g., stainless steel, MP35N, platinum, platinum-iridium alloy, etc.). As can be understood from FIG. 9, the extension 64 may be caused to mechanically contact the strip 44. For example, in the context of a metal electrode 24 or contact of the connector end 18 equipped with a barb 64, the bulk material surrounding and isolating the strip 44 at arrow B may not need to be removed prior to the electrode 24 being mounted on the tubular body 12, the barb 64 simply being pushed through the bulk material of the layer 31 to contact the strip 44 and place the strip and electrode in electrical communication. Alternatively, the extension 64 may be adhered to the strip 44 via an electrically conductive adhesive or epoxy once the bulk material is removed from over the strip 44 in the vicinity of the extension 64.

In one embodiment, the electrode 24 or contact of the connector end 18 may be formed of an electrically conductive non-metal, such as, for example, electrically conductive films, electrically conductive polymers (e.g., electrically conductive silicone rubber, hydrogel, etc.) or other materials printed, formed, molded, or otherwise deposited over the exposed strip 44.

With respect to the connector pins of the lead connector end 18, depending on the embodiment, conductive epoxies or adhesives may be employed to establish electrical contact between the connector pins and the respective strips 44. Alternatively, the lead connector end 18 could be molded onto the lead body proximal end. The connector end 18 may have wires or prongs extending from the lead connector end contact rings and contact pin to the appropriate respective strip 44 to establish electrical contact.

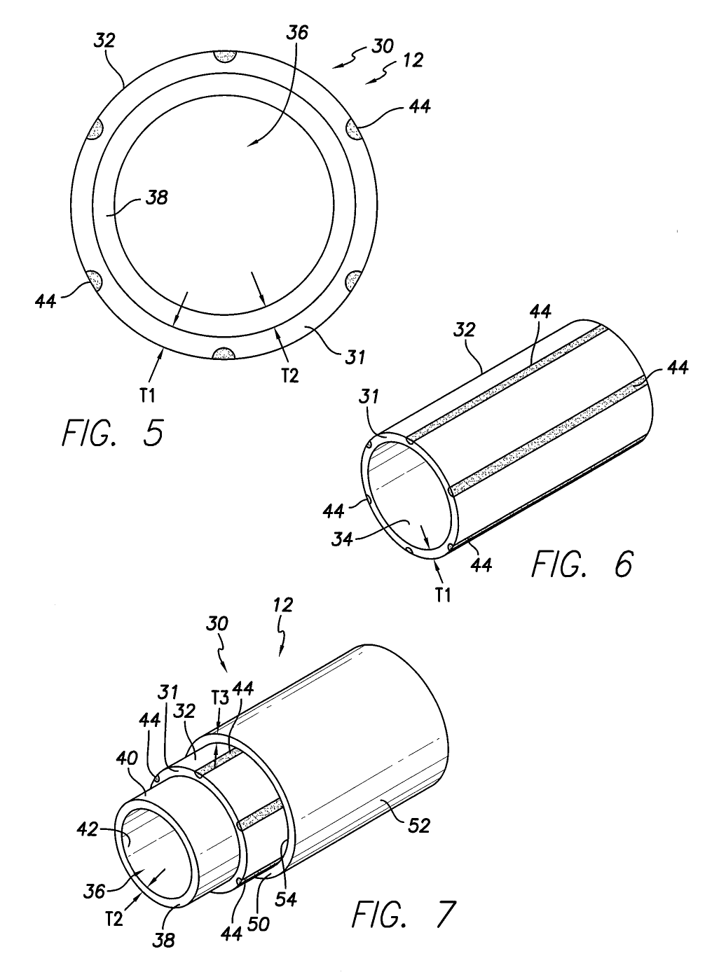

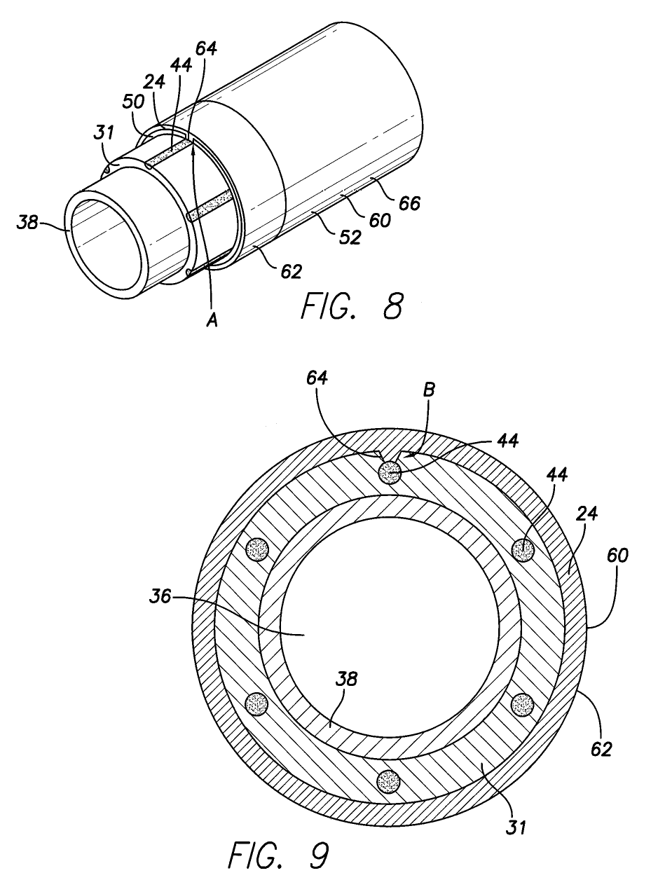

For a detailed discussion regarding a second configuration of the wall structure 30 of the tubular body 12, reference is made to FIGS. 5-7. FIG. 5 is a cross section of an intermediate layer 31 and an inner layer 38 of the tubular body 12 as taken along section line A-A in FIG. 1. FIG. 6 is an isometric view of the intermediate layer 31 of the tubular body in the same view as FIG. 5. FIG. 7 is an isometric view of the wall structure 30 of the tubular body in the same view as FIG. 7. As shown in FIG. 7, in some embodiments, the tubular body 12 includes a wall structure 30 including multiple layers, for example, a first or intermediate layer 31, a second or inner layer 38, and a third or outer layer 50. In other embodiments, the wall structure 30 may have a greater or lesser number of layers.

As shown in FIGS. 5-7, the intermediate layer 31 of the wall structure 30 may have an outer circumferential surface 32, an inner circumferential surface 34 and a thickness T1. The inner layer 38 of the wall structure 30 may be a helically wound coil, a braid layer, or a polymer layer formed of a polymer different from the polymer forming the intermediate layer 31. The inner layer 38 may include an outer circumferential surface 40, an inner circumferential surface 42, and a thickness T2. The inner circumferential surface 42 of the inner layer 38 may define the central lumen 36. The outer layer 50 of the wall structure 30 may have an outer circumferential surface 52, an inner circumferential surface 54 and a thickness T3. The outer layer 50 extends about the intermediate layer 31 and the intermediate layer 31 extends about the inner layer 38.

As shown in FIGS. 5-7, in one embodiment, the intermediate layer 31 includes electrical conductors 44 longitudinally extending through the thickness T1 of the intermediate layer 31. In one embodiment, the electrical conductors 44 are in the form of strips 44 of electrically conductive polymer material partially imbedded in the material forming the bulk of the intermediate layer 31 such that the strips 44 form a portion of the outer circumferential surface 32 of the intermediate layer 31. Thus, in one embodiment, the electrically conductive polymer strips 44 may be exposed along the entirety of their respective routes along the intermediate layer 31 were it not for the outer layer 50 that extends about the intermediate layer 31.

In one embodiment, the intermediate layer 31 may be formed of PEBAX with the electrically conductive polymer strips 44 being coextruded along with the PEBAX forming the intermediate layer 31. The electrically conductive polymer strips 44 may be formed of electrically conductive silicone rubber, epoxy, adhesive, etc. In other embodiments, the intermediate layer 31 may be formed of other materials besides PEBAX, for example, polyurethane, SPC, nylon, etc. In some embodiments, the intermediate layer 31 or any of the rest of the layers 38, 50 may be formed of multiple layers.

In one embodiment, as indicated in FIG. 7, the intermediate layer 31 with its integral coextruded electrically conductive polymer strips 44 may be extruded over the inner layer 38 or coextruded with the inner layer 38, wherein the inner layer 38 may be, for example, a PEBAX layer, a PTFE inner tube, a braided layer, or etc. The outer layer 50 may then be extruded over the combined inner and intermediate layers 38, 31 or, alternatively, pulled over the combined inner and intermediate layers 38, 31 and then subjected to a reflow process as described above. The outer layer 50 may be formed of PEBAX, polyurethane, SPC, nylon, etc.

In one embodiment, the three layers 31, 38, 50 may be coextruded together.

To access the conductive strips 44, for example, to allow for an electrical connection between the strips 44 and an electrode 24 or contact ring of a connector end 18, the PEBAX of the outer layer 50 may be cut away (e.g., via mechanical, laser, chemical or other cutting processes) over the strips 44 in those areas needed to allow for the electrical connection.

In one embodiment, as depicted in FIG. 8, which is generally the same view as depicted in FIG. 7, once the wall structure 30 of the tubular body 12 is assembled as discussed with respect to FIGS. 5-7, a portion of the thickness T3 (compare FIGS. 7 and 8) is removed from the outer layer 50 to accommodate the placement of an electrode 24 or contact ring of a connector end 18 in a configuration where the tubular body 12 is generally isodiametric, the outer circumferential surface 60 of the lead body 12 being generally continuous and consistent with respect to diameter. In the vicinity of one of the electrically conductive strips 44, the outer layer 50 is removed in its entirety, as indicated by arrow A. In such an embodiment, the outer circumferential surface 52 (see FIG. 7) of the outer layer 50 in combination with the outer circumferential surface 62 of the electrode 24 may form the outer circumferential surface 60 (see FIG. 8) of the tubular body 12. Also, the remaining portion of the outer layer 50 may serve to electrically isolate the electrode 24 from all of the strips 44, except the strip 44 exposed at arrow A, which may be in electrical contact with the strip 44 via an extension 64 of the electrode 24 that extends through the outer layer 50 to contact the strip 44.

In another embodiment, as can be understood from FIG. 8, the wall structure 30 of the tubular body 12 is assembled as described above with respect to FIG. 7. The outer layer 50 remains in its entirety, except in a location over one of the strips 44, as indicated by arrow A. An electrode 24 is mounted over the outer circumferential surface of the outer layer 50 such that the outer layer 50 electrically isolates the electrode 24 from all of the strips 44, except the exposed strip 44 at arrow A, which is in electrical contact with the extension 64 of the electrode 24 that extends through the opening made in the outer layer 50. To make the tubular body 12 generally isodiametric, another layer 66 may be extended about the outer layer 50 generally everywhere not occupied by the electrode 24, the outer circumferential surfaces of the electrode 24 and the another layer 66 forming the outer circumferential surface 60 of the tubular body 12 and acting as a jacket 66. In such an embodiment, the jacket layer 66 may be formed of silicone rubber, SPC, polyurethane, etc.

In one embodiment, the electrode 24 or contact of the connector end 18 may be in the form of a ring, partial ring, button or other configuration. The electrode 24 or contact of the connector end 18 may be formed of an electrically conductive metal (e.g., stainless steel, MP35N, platinum, platinum-iridium alloy, etc.). As can be understood from FIG. 8, the extension 64 may be caused to mechanically contact the strip 44. For example, in the context of a metal electrode 24 or contact of the connector end 18 equipped with a barb 64, the bulk material surrounding and isolating the strip 44 at arrow A may not need to be removed prior to the electrode 24 being mounted on the tubular body 12, the barb 64 simply being pushed through the bulk material of the layer 31 to contact the strip 44 and place the strip and electrode in electrical communication. Alternatively, the extension 64 may be adhered to the strip 44 via an electrically conductive adhesive or epoxy once the bulk material is removed from over the strip 44 in the vicinity of the extension 64.

In one embodiment, the electrode 24 or contact of the connector end 18 may be formed of an electrically conductive non-metal, such as, for example, electrically conductive films, electrically conductive polymers (e.g., electrically conductive silicone rubber, hydrogel, etc.) or other materials printed, formed, molded, or otherwise deposited over the exposed strip 44.

As can be understood from FIG. 15, which is isometric views of the body proximal end 70 and a connector end 18, the configuration provided by the strips 44 may be employed to facilitate a method of attaching the connector end 18. The connector end 18 may have a number of connector pins 72 having an arrangement that generally matches the arrangement of the strips 44. In such an embodiment, the lead connector end 18 could be molded onto the body proximal end 70 with the strips 44 and pins 72 aligned. Alternatively, in one embodiment, the connector end 18 may have wires or prongs 74 extending from the connector pins 72 to be inserted into the strips 44 once the strips 44 and prongs 74 are aligned. Alternatively, conductive epoxies or adhesives may be employed to establish electrical contact between the connector pins 72 and the respective strips 44.

As can be understood from FIGS. 5-8, in an alternative embodiment, the outer layer 50 may be formed of a non-conductive epoxy or adhesive, which extends over the strips 44 of the intermediate layer 31 to electrically isolate the strips 44 in a manner similar to that provided if the outer layer 50 were formed of a PEBAX or other polymer layer. The epoxy or adhesive outer layer 50 may eliminate the cutting or removal step involved with exposing the strips 44 for connecting to the electrodes 24 as the epoxy or adhesive outer layer 50 may be applied in generally any desired pattern. In other words, the epoxy or adhesive outer layer 50 could be applied such that an opening in the layer 50 is provided where needed for connecting the strip 44 to the electrode 24.

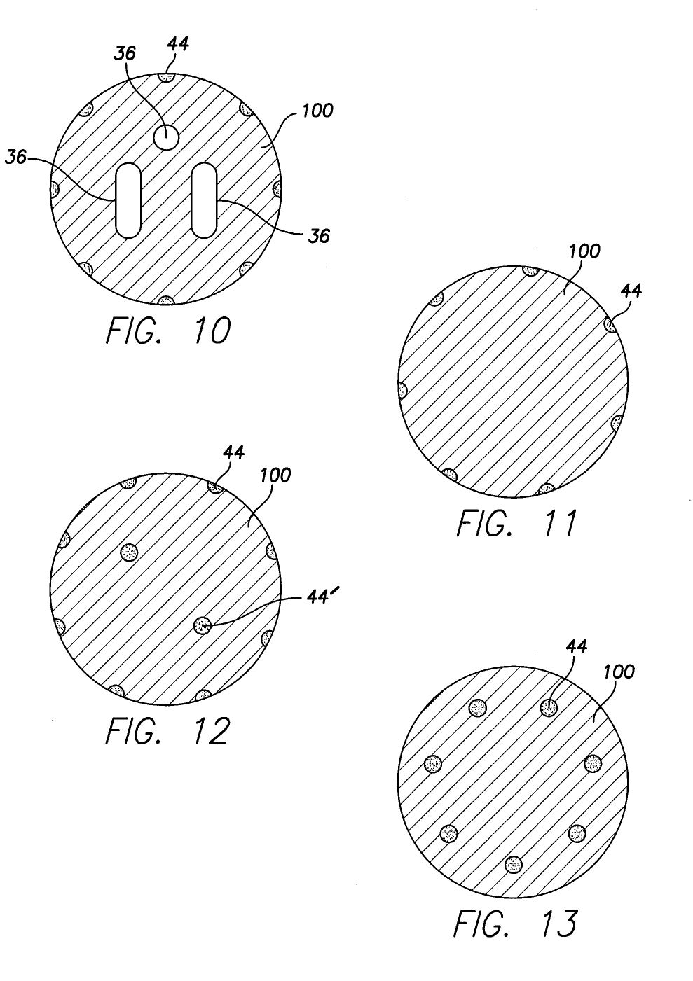

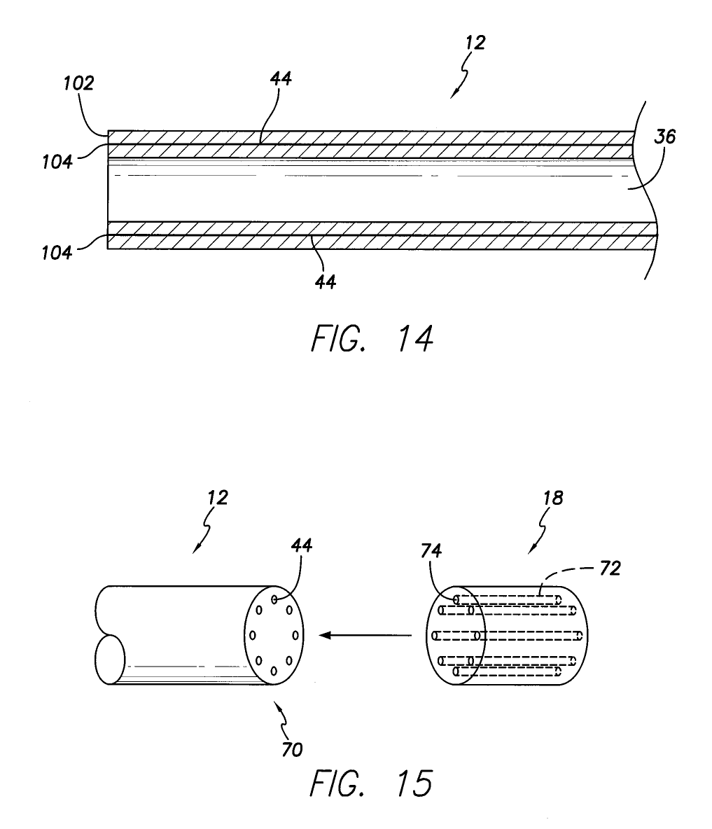

While the embodiments depicted in FIGS. 2-9 depict the body 12 as being tubular and having a central lumen 36 extending along the longitudinal axis of the body 12, in other embodiments the body 12 may be a generally solid core 100. As shown in FIGS. 10-13, which are cross sections similar to FIG. 2, the solid core 100 may be formed of a polymer material such as, for example, PTFE, ethylene tetrafluoroethylene (“ETFE”), PEBAX, polyurethane, SPC, silicone rubber, etc. As shown in FIGS. 10-13, the core 100 may have strips 44 imbedded in the core material and partially exposed as discussed with respect to the intermediate layer 31 of FIGS. 5-8. As depicted in FIGS. 12 and 13, the core 100 may also have strips 44′ that are completely imbedded in the core material such that the strips 44′ are not exposed, similar to that discussed with respect to the first layer of FIGS. 2-4.

As indicated in FIG. 10, the core 100 may have other types of lumens 36 in place of a central lumen 36 shown in FIGS. 2-9. For example, the lumens 36 may be located anywhere within the cross section of the core 100, including offset from the longitudinal axis of the body 12. Also, the lumens 36 may have cross sections that are circular, elliptical or other shape types, and there may be any number of lumens 36.

The strips 44 may be coextruded with the rest of the material forming the core 100. Depending on the embodiment, the outer circumferential surface of the core 100 may have an outer layer or coating as described above with respect to FIGS. 5-7, and electrodes may be mounted on the core 100 and electrically coupled to the strips 44 as described above with respect to FIGS. 5-7.

As shown in FIG. 14, which is a longitudinal cross section of the tubular body 12, the strips may extend through the wall thickness of the tubular body 12 and be exposed at the distal end 102 of the tubular body 12. Thus, the exposed ends 104 of the strips 44 may form conductive electrode tips 104.

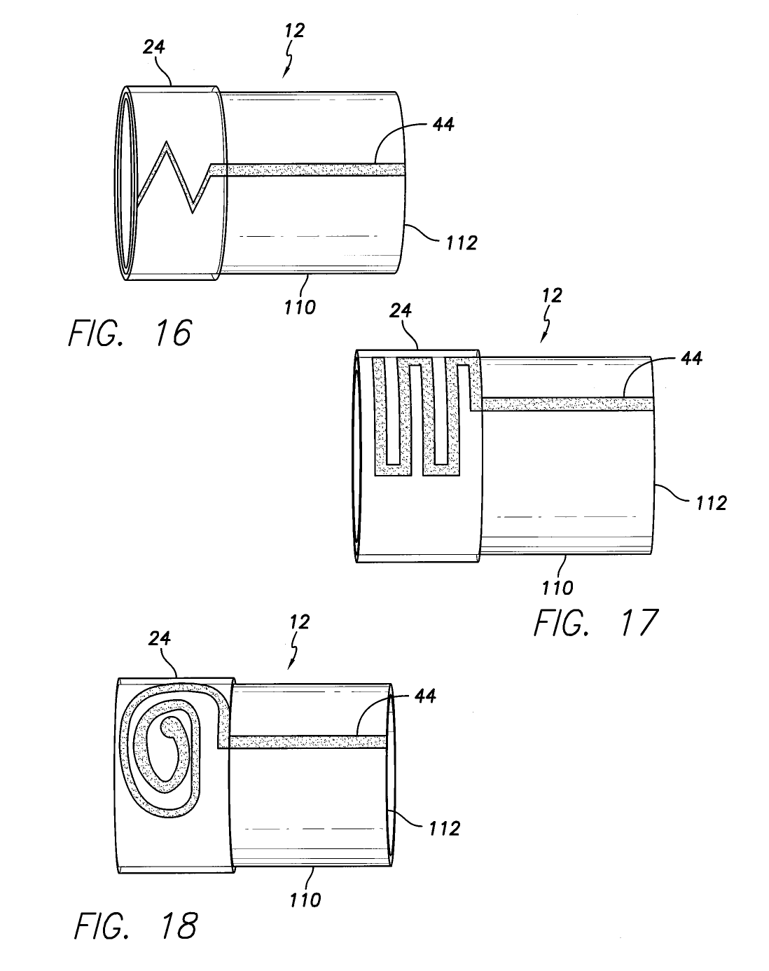

While the embodiments discussed above with respect to FIGS. 2-15 depict conductive polymer strips 44 imbedded in a polymer material forming at least a layer of the body 12, in other embodiments, the strips 44 may be an electrically conductive deposition on a surface of a layer forming the body 12. For example, as shown in FIGS. 16-18, which are isometric views of the body 12, an outer circumferential surface 110 of a layer 112 of the body 12 may have strip 44 in the form of an electrically conductive ink or other material may be deposited on the surface 110 vapor deposition or other methods. Because of the strips 44 being deposited via a deposition process, the strips 44 may be provided in a wide variety of patterns, as indicated in FIGS. 16-18. Such patterns may facilitate the electrical contact between electrodes 24 and the strips 44 by increasing the area for electrical contact. Electrical insulation layers may be provided over the outer circumferential surface 110 and strips 44 via extruding an electrically insulating polymer layer (e.g., PEBAX) over the surface 110 and strips 44, spraying, painting or otherwise depositing an electrically insulating epoxy or adhesive over the surface 110 and strips 44, or printing an electrically insulating ink over the surface 110 and strips 44. Electrically connecting the electrodes 24 to the strips 44 may then be accomplished via any of the methods discussed above with respect to FIGS. 2-9.

Although the present invention has been described with reference to preferred embodiments, persons skilled in the art will recognize that changes may be made in form and detail without departing from the spirit and scope of the invention.

What is claimed is:

1. A medical tubular body comprising:
a tubular layer formed of an electrically insulating polymer and an electrically conductive polymer strip imbedded in and longitudinally extending through the insulating polymer.

2. The tubular body of claim 1, wherein the strip is completely imbedded in the insulating polymer.

3. The tubular body of claim 1, wherein the strip is not completely imbedded in the insulating polymer, the strip forming a portion of an outer circumferential surface of the tubular layer along with the insulating polymer.

4. The tubular body of claim 3, further comprising an electrically insulating layer extending about the outer circumferential surface.

5. The tubular body of claim 4, wherein the insulating layer is a second electrically insulating polymer.

6. The tubular body of claim 5, wherein the second insulating polymer includes at least one of PEBAX, polyurethane, SPC, and silicone rubber.

7. The tubular body of claim 4, wherein the insulating layer includes at least one of an electrically insulating adhesive, an electrically insulating epoxy, and an electrically insulating ink.

8. The tubular body of claim 3, further comprising an electrode that is at least one of molded, sprayed and vapor deposited against the polymer strip.

9. The tubular body of claim 1, wherein the polymer strip includes at least one of silicone rubber, epoxy, and adhesive.

10. The tubular body of claim 1, wherein the insulating polymer includes at least one of PEBAX, polyurethane, SPC, and silicone rubber.

11. The tubular body of claim 1, further comprising an electrode that includes a portion that extends into the polymer strip.

12. The tubular body of claim 11, wherein the electrode is an electrically conductive metal.

13. The tubular body of claim 1, further comprising an electrode that includes a portion that extends through a portion of the insulating polymer to electrically contact the polymer strip.

14. The tubular body of claim 1, wherein the medical tubular body is at least one of an implantable medical lead, a sheath, a catheter, and an introducer.

15. A medical longitudinally extending body comprising:
a longitudinally extending portion of the body, the longitudinally extending portion formed of an electrically insulating polymer and an electrically conductive polymer strip imbedded in and longitudinally extending through the insulating polymer, the insulating polymer forming a majority of the longitudinally extending portion.

16. The body of claim 15, wherein the strip is at least one of completely imbedded in the insulating polymer.

17. The body of claim 15, wherein the strip is not completely imbedded in the insulating polymer, the strip forming a portion of an outer circumferential surface of the insulating polymer.

18. The body of claim 15, wherein the longitudinally extending portion is a tubular layer.

19. The body of claim 15, wherein the longitudinally extending portion is a generally solid cylindrical body.

20. The body of claim 19, wherein the body includes multiple longitudinally extending lumens.

21. The body of claim 15, wherein the medical longitudinally extending body is at least one of an implantable medical lead, a sheath, a catheter, and an introducer.

22. A medical longitudinally extending body comprising:
a longitudinally extending portion of the body, the longitudinally extending portion formed of an electrically insulating polymer; and
an electrically conductive strip extending along an outer circumferential surface of the longitudinally extending portion of the body, wherein the strip is deposited via at least one of vapor deposition, printing, and painting.

23. The body of claim 22, wherein the strip is an electrically conductive ink.

24. The body of claim 22, wherein the longitudinally extending portion is a tubular layer.

25. The body of claim 22, wherein the longitudinally extending portion is a generally solid cylindrical body.

26. The body of claim 25, wherein the body includes multiple longitudinally extending lumens.

27. The body of claim 22, further comprising an electrically insulating layer extending about the outer circumferential surface.

28. The body of claim 27, wherein the electrically insulating layer is at least one of a polymer layer, an ink, an epoxy, and an adhesive.

29. The body of claim 22, wherein the medical longitudinally extending body is at least one of an implantable medical lead, a sheath, a catheter, and an introducer.

30. A method of manufacturing a medical longitudinally extending body, the method comprising:
providing an electrically insulating polymer;
providing an electrically conductive polymer; and
co-extruding the electrically insulating polymer and the electrically conductive polymer into a longitudinally extending portion of the medical longitudinally extending body;
wherein the insulating polymer forms a majority of the longitudinally extending portion and the electrically conductive polymer forms a strip imbedded in and longitudinally extending through the insulating polymer.

31. The method of claim 30, wherein the strip is at least one of completely imbedded in the insulating polymer.

32. The method of claim 30, wherein the strip is not completely imbedded in the insulating polymer, the strip forming a portion of an outer circumferential surface of the insulating polymer.

33. The method of claim 30, wherein the longitudinally extending portion is a tubular layer.

34. The method of claim 33, further comprising at least one of pulling the tubular layer over and extruding the tubular layer over another longitudinally extending portion of the medical longitudinally extending body.

35. The method of claim 30, wherein the longitudinally extending portion is at least one of a tubular body and a generally solid cylindrical body.

36. The method of claim 36, further comprising providing an electrical insulating layer over the longitudinally extending portion.

37. The method of claim 30, further comprising causing the medical longitudinally extending body to be at least one of an implantable medical lead, a sheath, a catheter, and an introducer.
